(19) United States
(12) Patent Application Publication
(10) Pub. No.: US 2023/0349778 A1
(43) Pub. Date: Nov. 2, 2023

(54) WEARABLE AND HIGHLY STRETCHABLE STRAIN GAUGE USING PEDOT:PSS/WPU CONDUCTIVE POLYMER COMPOSITE APPLICABLE TO VARIOUS BIOMEDICAL DEVICES AND ELECTRONIC DEVICES, AND METHOD OF MANUFACTURING THEREOF

(71) Applicant: NATIONAL TSING HUA UNIVERSITY, HSINCHU (TW)

(72) Inventor: Wei-Chih WANG, Sammamish, WA (US)

(21) Appl. No.: 17/731,518

(22) Filed: Apr. 28, 2022

(51) Int. Cl. G01L 1/22; G01L 5/1627; A61B 5/257; A61B 5/28; A61F 13/00

(52) U.S. Cl. CPC G01L 1/2287; G01L 5/1627; A61B 5/257; A61B 5/28; A61F 13/00051; A61B 2562/0261

(57) ABSTRACT

According to the present disclosure, a conductive polymer composite and a strain gauge are provided. The conductive polymer composite includes poly(3,4-ethylenedioxythiophene): polystyrene sulfonate and waterborne polyurethane, and the conductive polymer composite is homogeneous. The strain gauge includes a substrate and a strain sensitive layer. The substrate has a surface, and the strain sensitive layer is connected to the surface of the substrate. The strain sensitive layer is made of the aforementioned conductive polymer composite, and the strain sensitive layer has at least four separations arranged in a staggered way and forms bow-like structures, which makes the strain sensitive layer deform more in a first direction than a second direction perpendicular to the first direction.

### BACKGROUND

### Technical Field

The present disclosure relates to a conductive polymer composite, a strain gauge and a method of manufacturing thereof. More particularly, the present disclosure relates to a strain gauge, which is made of a conductive polymer composite and applicable to biomedical devices and electronic devices, and a method of manufacturing thereof.

### Description of Related Art

Strain gauge is an electrical sensor for measuring force or strain of an object. Strain gauge has been widely used in all kinds of industries with various material and technologies used, and metals and semiconductors are the main used materials of the traditional strain gauge. However, metal strain gauge has many disadvantages, such as high-cost, complex manufacturing process and possible toxicity. Metal strain gauge and semiconductor strain gauge are unable to measure large strain and are not suitable for human body test due to the stiff properties thereof.

Other conducting materials used as strain gauge are poly(3,4-ethylenedioxythiophene) (PEDOT) or poly(3,4-ethylenedioxythiophene): polystyrene sulfonate (PEDOT:PSS). PEDOT strain gauge is proven to be comparable to commercial available strain gauge, but PEDOT strain gauge has poor processability because the processing of PEDOT strain gauge still relies on micro-fabrication and polymerization, which requires a higher fabrication cost and more processing time. On the other hand, PEDOT:PSS strain gauge has process benefit by inkjet printing or molding, but has reliability issue in large strain measurement. The material is brittle. The Young's modulus of PEDOT:PSS material changes depends on humidity, and the strain limit of PEDOT:PSS material is around 0.1 strain, which limits the application of PEDOT:PSS strain gauge in biomedical field.

In this regard, the scientists are still aiming to develop a conductive material which performs well in strain measurement in biomedical area.

### SUMMARY

According to one aspect of the present disclosure, a conductive polymer composite includes poly(3,4-ethylenedioxythiophene):polystyrene sulfonate and waterborne polyurethane, and the conductive polymer composite is homogeneous.

According to another aspect of the present disclosure, a strain gauge includes a substrate and a strain sensitive layer. The substrate has a surface, and the strain sensitive layer is connected to the surface of the substrate. The strain sensitive layer is made of the conductive polymer composite of the aforementioned aspect, and the strain sensitive layer has at least four separations arranged in a staggered way and forms bow-like structures, which makes the strain sensitive layer deform more in a first direction than a second direction perpendicular to the first direction.

According to one another aspect of the present disclosure, a biomedical device includes the strain gauge of the aforementioned aspect, and the biomedical device is a smart bandage or an ECG pad.

According to still another aspect of the present disclosure, an electronic device includes the strain gauge of the aforementioned aspect, and the electronic device is a humidity sensor, a touch sensor, a touch screen or a shear sensor.

According to still another aspect of the present disclosure, a method of manufacturing a strain gauge includes steps as follows. A substrate is provided, an etching step is performed and a coating step is performed. The substrate has a surface, and a pattern is etched on the surface of the substrate in the etching step. In the coating step, the conductive polymer composite of the aforementioned aspect is coated onto the surface, which is etched, of the substrate, so as to form a strain sensitive layer on the substrate, and the strain gauge is obtained. The strain sensitive layer has at least four separations arranged in a staggered way and forms bow-like structures, which makes the strain sensitive layer deform more in a first direction than a second direction perpendicular to the first direction.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1A is a three-dimensional schematic view of a strain gauge according to one embodiment of the present disclosure.

FIG. 1B is a top schematic view of the strain gauge of FIG. 1A.

FIG. 1C is a side schematic view of the strain gauge of FIG. 1A.

FIG. 2 is a flow chart of one embodiment of a method of manufacturing the strain gauge according to the present disclosure.

FIG. 3 is a flow chart of another embodiment of the method of manufacturing the strain gauge according to the present disclosure.

FIG. 4A is a resistance and strain curve diagram of the strain gauge of the 1st comparison.

FIG. 4B is a resistance and strain curve diagram of the strain gauge of the 1st example.

FIG. 5A is a strain distribution of the strain gauge of the 2nd comparison.

FIG. 5B is a strain distribution of the strain gauge of the 2nd example.

FIG. 5C is a strain distribution of the strain gauge of the 3rd example.

FIG. 6A is a relationship diagram between the number of cells of a strain sensitive layer of the strain gauge and maximum strain thereof.

FIG. 6B is a relationship diagram between the number of cells of the strain sensitive layer of the strain gauge and effective Poisson's ratio thereof.

FIG. 7A is a relationship diagram between strain and resistance of the strain gauge of the 4th example.

FIG. 7B is a relationship diagram between tensile force and resistance of the strain gauge of the 4th example.

FIG. 7C is a relationship diagram between strain and tensile force of the strain gauge of the 4th example.

FIG. 8 is a relationship diagram between strain and force in a fatigue test of the strain gauge of the 4th example.

FIG. 9 is a schematic view of the strain gauge of the 4th example which stretches to 56% in strain.

FIG. 10 is a schematic view of a bending test on human back using the strain gauge of the 5th example.

FIG. 11 is a resistance changing diagram of the strain gauge of the 5th example in the bending test on human back.

### DETAILED DESCRIPTION

The present disclosure will be further exemplified by the following specific embodiments. However, the embodiments can be applied to various inventive concepts and can be embodied in various specific ranges. The specific embodiments are only for the purposes of description, and are not limited to these practical details thereof.

According to one aspect of the present disclosure, a conductive polymer composite includes poly(3,4-ethylenedioxythiophene):polystyrene sulfonate (PEDOT:PSS) and waterborne polyurethane (WPU), and the conductive polymer composite is homogeneous. PEDOT:PSS provides conductivity, and the mechanical strength of the conductive polymer composite is significantly enhanced by adding WPU. Also, the PEDOT:PSS and WPU can be easily dispersed in water solution and mixed well with PSS presented.

Furthermore, a ratio of PEDOT:PSS to WPU can be 4.5:1-6:1, so as to make the viscosity of the conductive polymer composite low and that the conductive polymer composite can be used in inject printing process. A ratio of poly(3,4-ethylenedioxythiophene) to polystyrene sulfonate can be 0.05-1.00 for desired morphology and physical properties of the conductive polymer composite.

The conductive polymer composite can further include dimethyl sulfoxide (DMSO), and a mass fraction of DMSO can be 2 wt. %-6 wt. %. With DMSO presented, the conductivity of the conductive polymer composite can be improved.

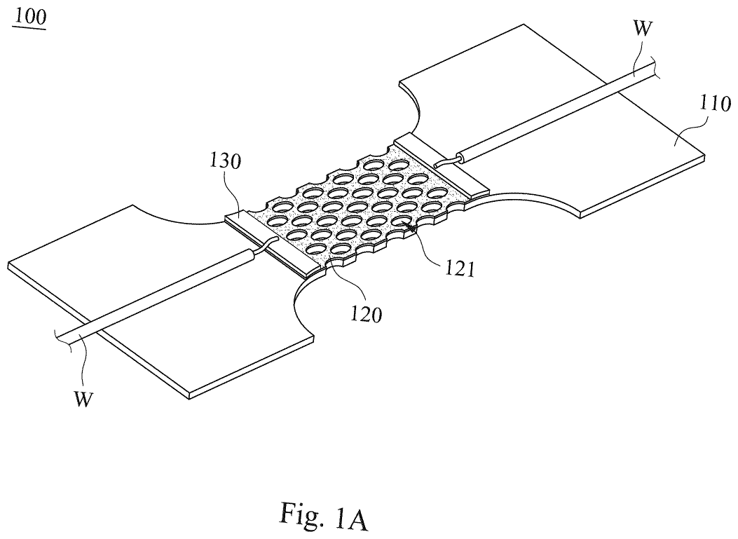

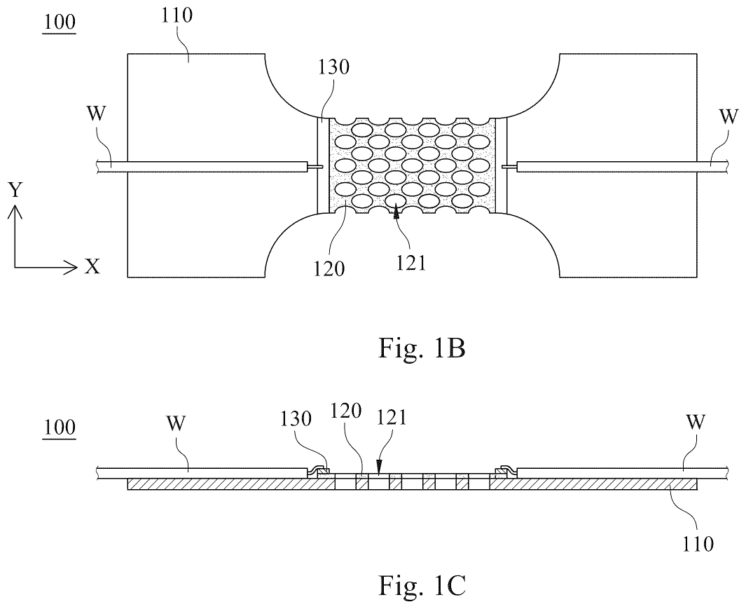

Please refer to FIG. 1A, FIG. 1B and FIG. 1C. FIG. 1A is a three-dimensional schematic view of a strain gauge 100 according to one embodiment of the present disclosure. FIG. 1B is a top schematic view of the strain gauge 100 of FIG. 1A. FIG. 1C is a side schematic view of the strain gauge 100 of FIG. 1A. According to another aspect of the present disclosure, the strain gauge 100 includes a substrate 110 and a strain sensitive layer 120 connected to a surface of the substrate 110, and the strain gauge 100 can perform a strain measurement and a torque measurement.

In detail, the substrate 110 can be made of an elastomer, and the strain sensitive layer 120 is made of the conductive polymer composite of the aforementioned aspect. The strain gauge 100 can further include a wire W, which is electrically connected to the strain sensitive layer 120. The wire W can be electrically connected to the strain sensitive layer 120 through a connector 130, which can be a magnetic connector, pre-soldered wire to copper tape or a silver epoxy. The strain gauge 100 can further include an adhesion layer disposed between the substrate 110 and the strain sensitive layer 120, wherein the adhesion layer can be made of WPU. The strain sensitive layer 120 has at least four separations 121 arranged in a staggered way and forms bow-like structures, which make the strain sensitive layer 120 deform more in a first direction Y than a second direction X perpendicular to the first direction Y.

Please refer to FIG. 1B. Each of the bow-like structures of the strain sensitive layer 120 has a central rigid anchor point to take up all the load while allowing the four long tapering elastic arms to stretch, so that the overall strain gauge 100 can be expand more easily.

Furthermore, an area of the separations 121 is A_{H}, an area of the strain sensitive layer 120 is A_{S}, and the following condition can be satisfied: 0.2≤A_{H}/A_{S}≤0.8. The strain sensitive layer 120 generates a first strain in the first direction Y and a second strain in the second direction X, and a difference between the first strain and the second strain increases as a number of the at least four separations 121 increases. Thus, the Poisson's ratio and localized strain of the strain gauge 100 are reduced because of the increasing separations 121, and it is favorable for the strain gauge 100 to measure the strain in a particular direction and a strain measurement of the strain gauge 100 can be up to 400% strain.

According to one another aspect of the present disclosure, a biomedical device includes the strain gauge 100 of the aforementioned aspect, and the biomedical device is a smart bandage or an ECG pad.

According to still another aspect of the present disclosure, an electronic device includes the strain gauge 100 of the aforementioned aspect, and the electronic device is a humidity sensor, a touch sensor, a touch screen or a shear sensor.

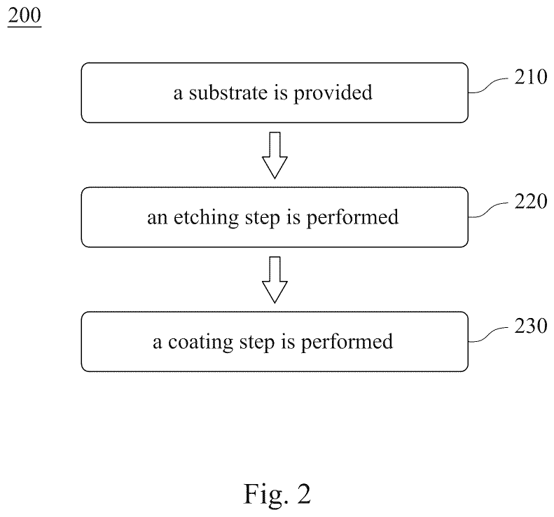

Please refer to FIG. 2. FIG. 2 is a flow chart of one embodiment of a method 200 of manufacturing the strain gauge according to the present disclosure. According to still another aspect of the present disclosure, the method 200 includes Step 210, Step 220 and Step 230.

In Step 210, a substrate, which can be made of an elastomer, is provided, and the substrate has a surface.

In Step 220, an etching step is performed by etching a pattern on the surface of the substrate, and the substrate can be etched by laser (e.g. UV laser, excimer laser, Nd:YAG laser, CO₂ laser, femtosecond laser) or stencil machine. Please refer back to FIG. 1A and FIG. 1B. The etched substrate in Step 220 can have the same structure as the substrate 110 of the aforementioned aspect, and the etched area of the substrate will become the area which is desired to be conductive after the following steps.

In Step 230, a coating step is performed to coat the conductive polymer composite of the aforementioned aspect onto the surface, which is etched, of the substrate, so as to form a strain sensitive layer on the substrate, and the strain gauge is obtained. The conductive polymer composite can be coated onto the surface by an inkjet printing method, a spreading method or a soaking method. Please note that, the structures and properties of the substrate, the strain sensitive layer and the strain gauge of the method 200 are the same as the substrate 110, the strain sensitive layer 120 and the strain gauge 100 of the aforementioned aspect, and the details will not be given herein.

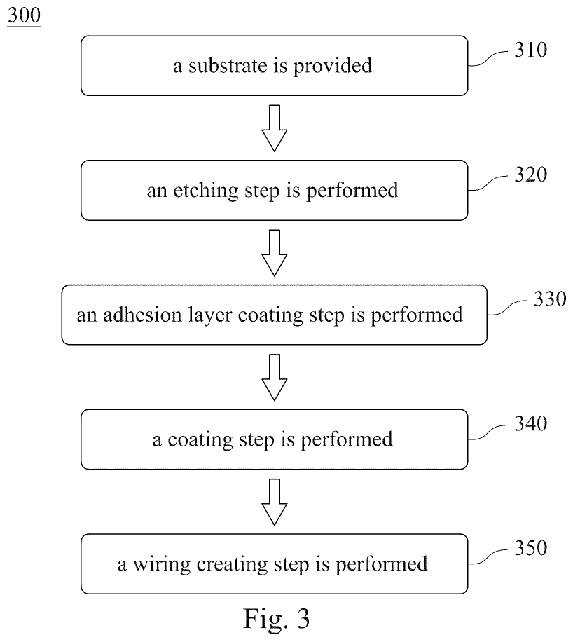

Please refer to FIG. 3. FIG. 3 is a flow chart of another embodiment of the method 300 of manufacturing the strain gauge according to the present disclosure. According to still another aspect of the present disclosure, the method 300 includes Step 310, Step 320, Step 330, Step 340 and Step 350.

In Step 310, a substrate is provided. In Step 320, an etching step is performed. In Step 340, a coating step is performed. The details of Step 310, Step 320 and Step 340 are the same as Step 210, Step 220 and Step 230 of the aforementioned aspect, and the details will not be given herein.

In Step 330, an adhesion layer coating step is performed to coat an adhesion layer onto the surface, wherein the adhesion layer is made of WPU. The adhesion layer is disposed between the substrate and the strain sensitive layer, so as to enhance the adhesion between the substrate and the strain sensitive layer. For example, the adhesion layer can be fabricated by spreading WPU on the substrate. After the spread WPU is dry, the conductive polymer composite can be coated on to the adhesion WPU layer and form the strain sensitive layer.

In Step 350, a wiring creating step is performed by applying a magnetic connector, pre-soldered wire to copper tape or a silver epoxy onto the strain sensitive layer, so as to form a wiring connection to the strain sensitive layer for transmitting electrical signals.

In the following part, mechanical hysteresis of the materials and mechanical and electrical properties of the strain gauges are tested, and the results thereof will be discussed.

<Mechanical Hysteresis Test>

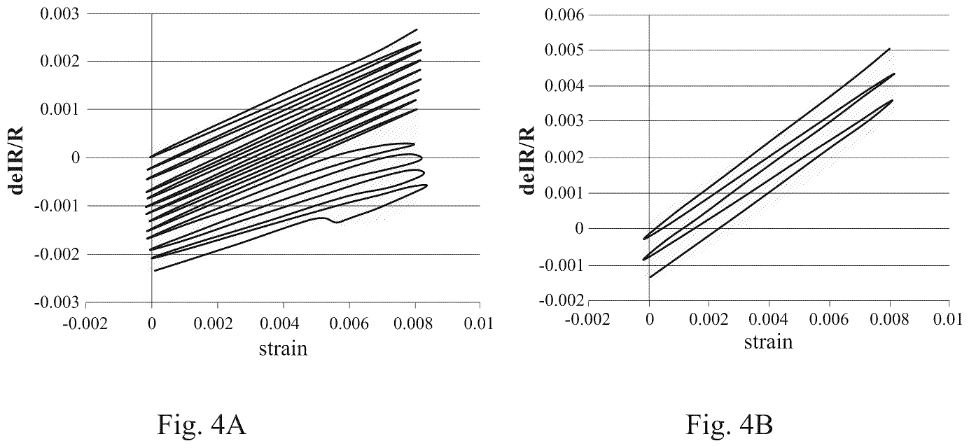

In this test, the mechanical hysteresis of the 1st comparison and the 1st example is compared. The 1st comparison is the strain gauge made of a PEDOT:PSS material, and the 1st example is the strain gauge made of the conductive polymer composite of the present disclosure. Please refer to FIG. 4A and FIG. 4B. FIG. 4A is a resistance and strain curve diagram of the strain gauge of the 1st comparison. FIG. 4B is a resistance and strain curve diagram of the strain gauge of the 1st example. In FIG. 4A and FIG. 4B, the mechanical hysteresis of the 1st comparison is much more severe than the 1st example, which means that the hysteresis in pristine PEDOT:PSS material can be significantly reduced by adding more mechanically elastic WPU.

<Strain Distribution Test>

In this test, the strain distributions of strain gauges of the 2nd comparison, the 2nd example and the 3rd example are compared. The 2nd comparison is the strain gauge with 2 separations, the 2nd example is the strain gauge with 8 separations, and the 3rd example is the strain gauge with 128 separations. Furthermore, in the following analysis, the structures of the strain gauges are simplified by calculating the number of cells. That is, the structure of the strain gauge of the 2nd comparison is taken as one cell, while the structures of the strain gauges of the 2nd example and the 3rd example are 4 cells and 64 cells, respectively.

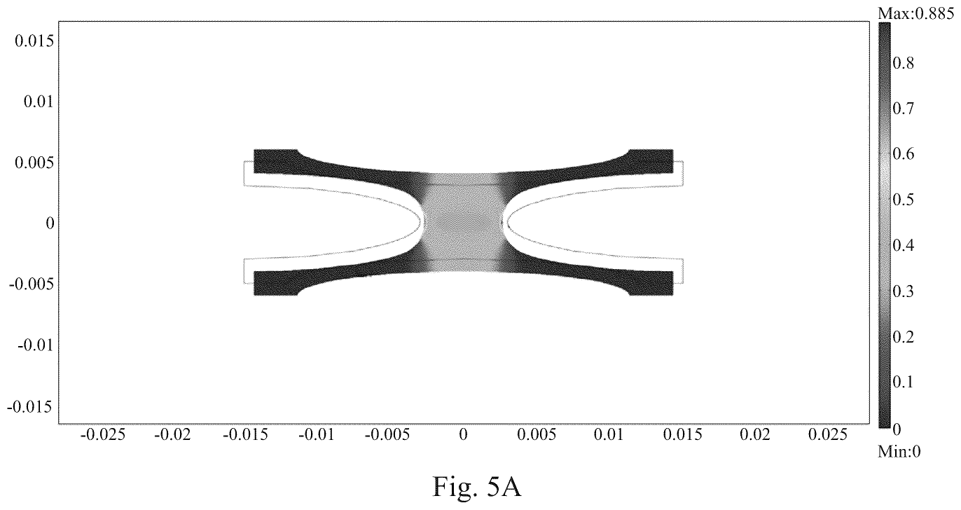

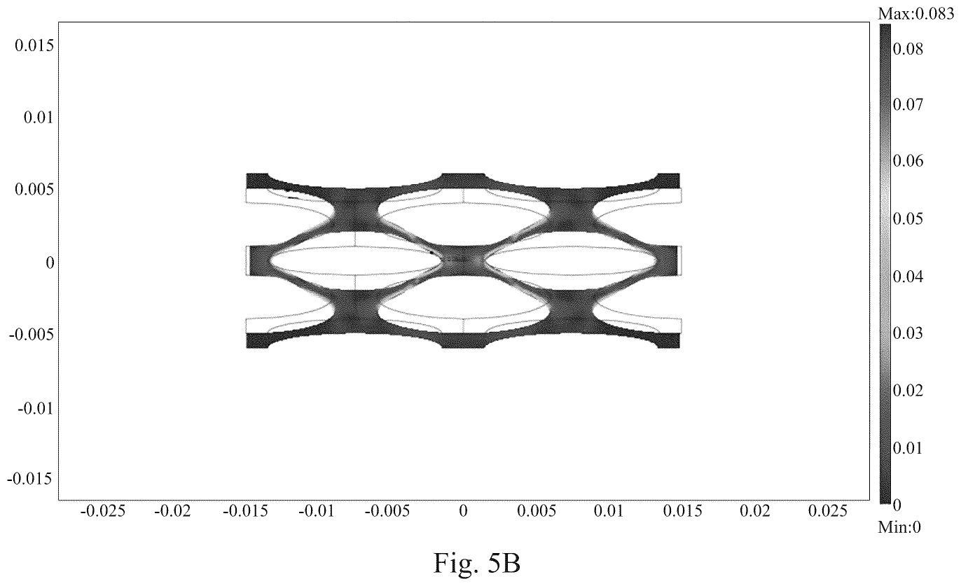

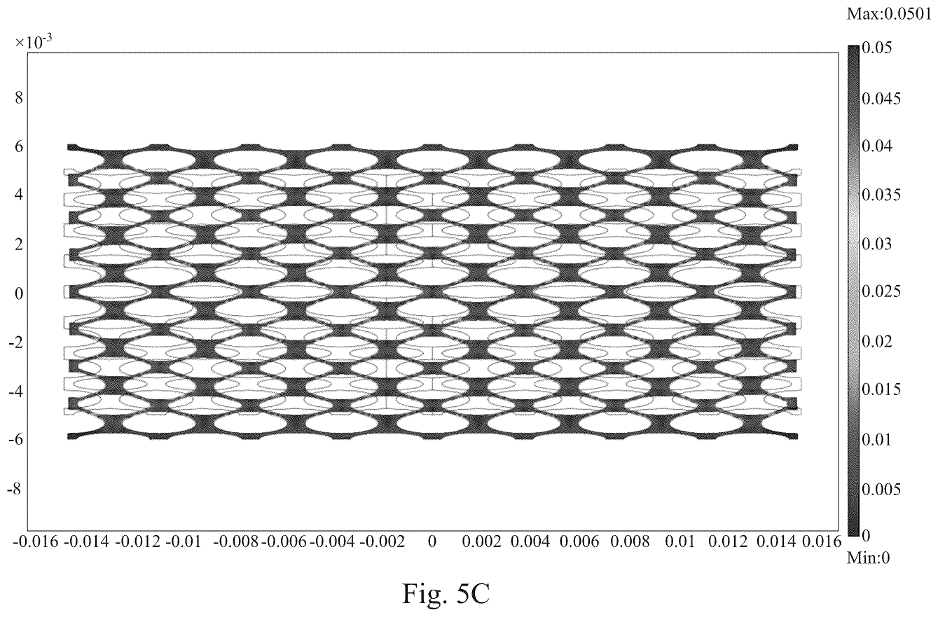

Please refer to FIG. 5A. FIG. 5A is a strain distribution of the strain gauge of the 2nd comparison. In FIG. 5A, it shows that the central rigid anchor point (the turquois color region) of the strain gauge takes up all the load while allowing the four long tapering elastic arms to stretch, so that the overall strain gauge can be expand more easily. The maximum strain of the strain gauge of the 2nd comparison is 88.5% and the effective Poisson's ratio thereof is 0.2161.

Please refer to FIG. 5B and FIG. 5C. FIG. 5B is a strain distribution of the strain gauge of the 2nd example. FIG. 5C is a strain distribution of the strain gauge of the 3rd example. The maximum strains of the strain gauges of the 2nd example and the 3rd example are 8.4% and 5.0%, respectively. The effective Poisson's ratios of the strain gauges of the 2nd example and the 3rd example are both around 0.06.

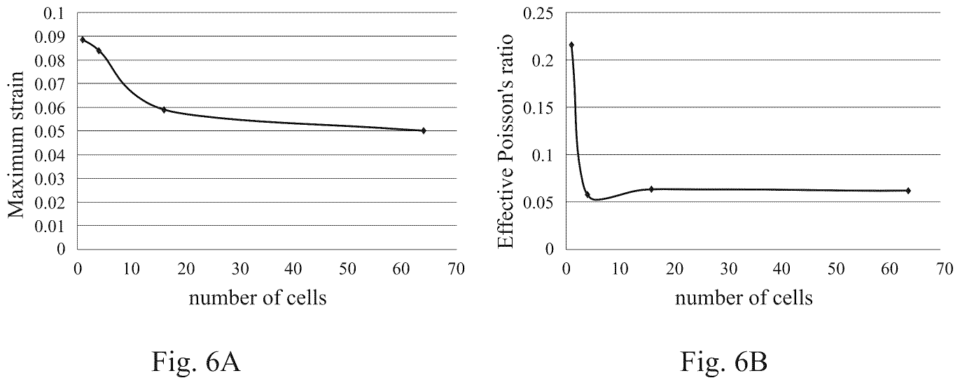

Please refer to FIG. 6A and FIG. 6B. FIG. 6A is a relationship diagram between the number of cells of the strain sensitive layer of the strain gauge and maximum strain thereof. FIG. 6B is a relationship diagram between the number of cells of the strain sensitive layer of the strain gauge and effective Poisson's ratio thereof. In FIG. 6A and FIG. 6B, it shows that the maximum strain of the strain gauge keeps decreasing as the number of cells increases, while the effective Poisson's ratio remains at around 0.06 for the strain gauges with more than four cells. According to FIG. 5B, FIG. 5C, FIG. 6A, FIG. 6B and the aforementioned results, it can be realized that the localized strain is further reduced and Poisson's ratio starts to settle at a very low value by increasing the number of cells/separations. Therefore, the strain gauge of the present disclosure is great for sensors that intend to measure only one direction strain.

<Mechanical and Electrical Properties>

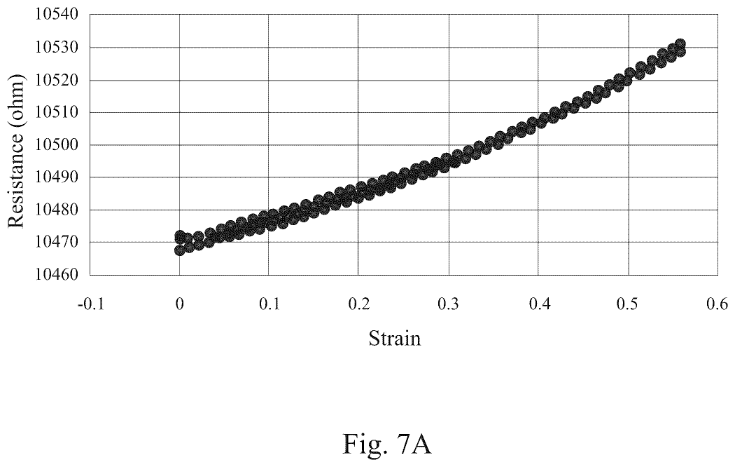

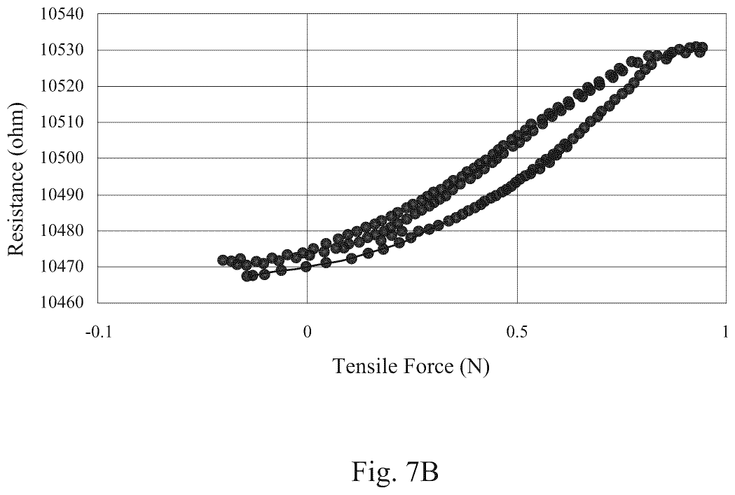

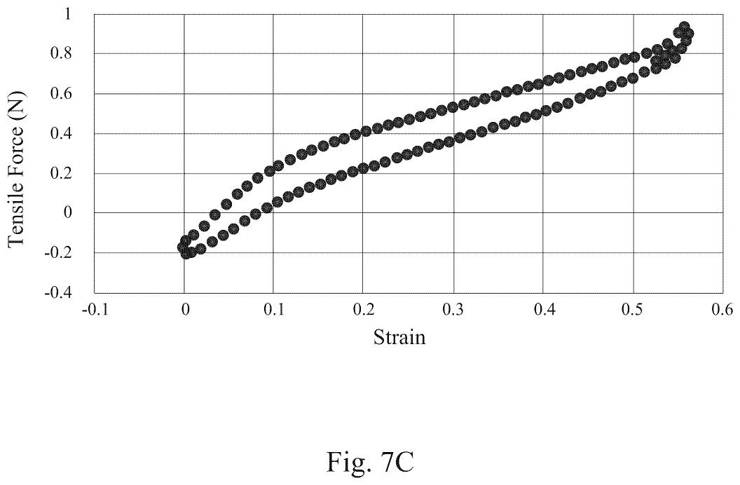

In this test, the relationships between strain, tensile force and resistance of the 4th example are found out. The 4th example is the strain gauge with 14 separations. Please refer to FIG. 7A, FIG. 7B and FIG. 7C. FIG. 7A is a relationship diagram between strain and resistance of the strain gauge of the 4th example. FIG. 7B is a relationship diagram between tensile force and resistance of the strain gauge of the 4th example. FIG. 7C is a relationship diagram between strain and tensile force of the strain gauge of the 4th example. In FIG. 7A and FIG. 7B, it shows that the resistance generated by the strain gauge is performed as a function of elongation and force of the strain gauge. In FIG. 7C, the strain of the strain gauge of the 4th example increases as the tensile force increases, which means the strain gauge of the present disclosure has a relatively elastic performance.

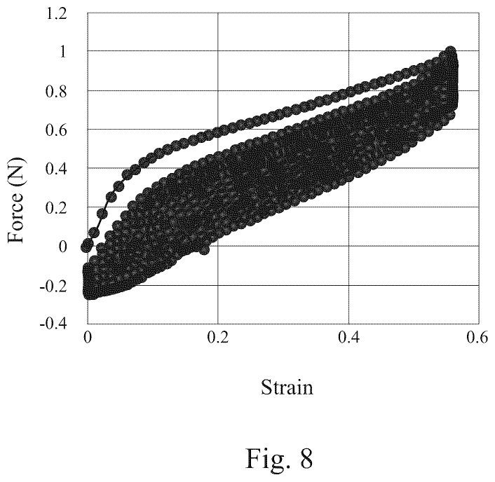

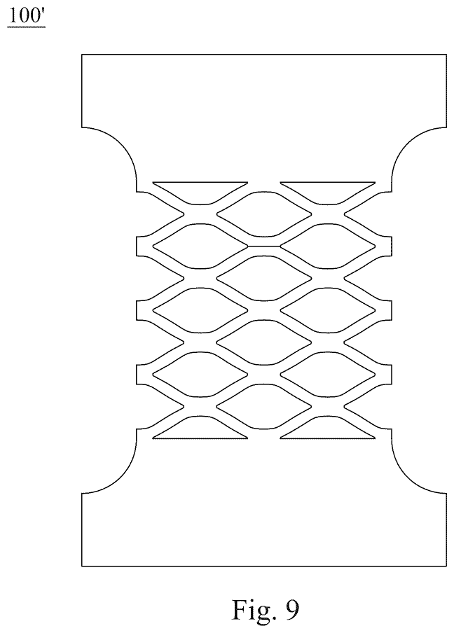

Please refer to FIG. 8. FIG. 8 is a relationship diagram between strain and force in a fatigue test of the strain gauge of the 4th example. In FIG. 8, it shows that the strain gauge of the 4th example is relatively robust and the strain changes are quite consistent in a long run. Furthermore, please refer to FIG. 9. FIG. 9 is a schematic view of the strain gauge 100′ of the 4th example which stretches to 56% in strain. In FIG. 9, it shows that the strain gauge of the present disclosure has great flexibility and large overall strain can be generated.

<Bending Test>

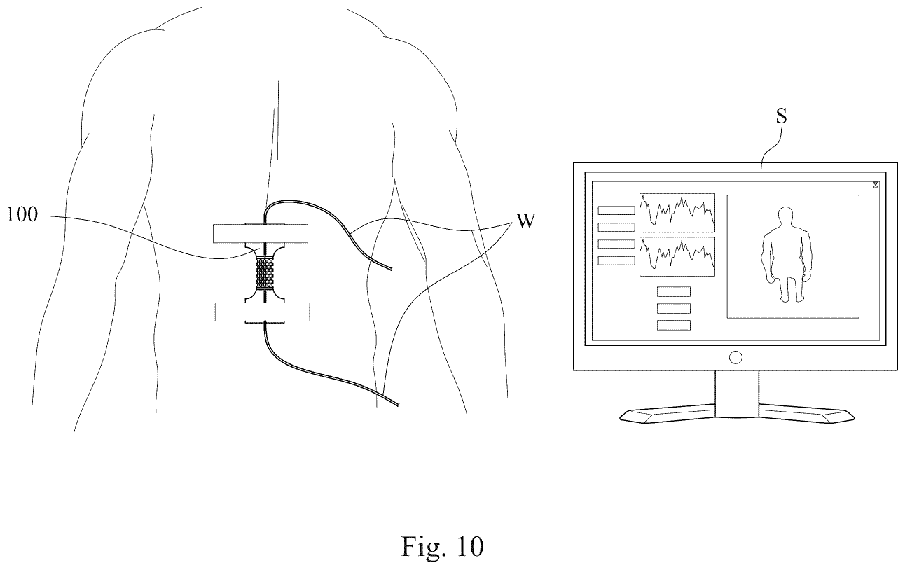

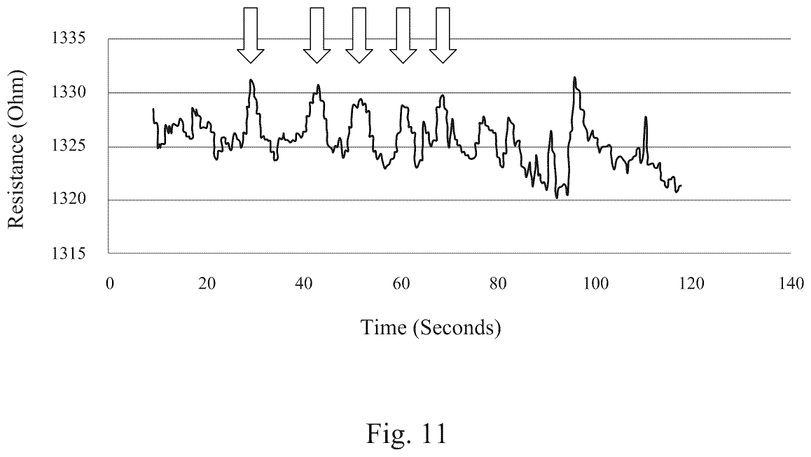

Please refer to FIG. 10. FIG. 10 is a schematic view of a bending test on human back using the strain gauge 100 of the 5th example. In this test, the strain gauge 100 of the 5th example is secured on a human back, and the wires W is connected to a testing system S, so as to transfer the electrical signals generates by the strain gauge 100 to the testing system S. Therefore, the resistance change as the human back bends is recorded. The strain gauge 100 of the 5th example includes 31 separations. Please refer to FIG. 11. FIG. 11 is a resistance changing diagram of the strain gauge 100 of the 5th example in the bending test on the human back. The arrows in FIG. 11 indicate when the human back is bending. Every time when the human back is bending, it forms a resistance peak on the diagram. Therefore, it shows that the strain gauge of the present disclosure is suitable for monitoring wound stress, heart rate or blood pressure without any additional embedded sensors.

According to the present disclosure, the conductive polymer composite with the characteristics of high processability, water solubility and flexibility is developed by introducing waterborne polyurethane into poly(3,4-ethylenedioxythiophene):polystyrene sulfonate. The strain gauge including the conductive polymer composite can perform large strain measurement with faster reaction, and is applicable to various sensing devices.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A conductive polymer composite, comprising:
poly(3,4-ethylenedioxythiophene):polystyrene sulfonate (PEDOT:PSS); and
waterborne polyurethane (WPU);
wherein the conductive polymer composite is homogeneous.

2. The conductive polymer composite of claim 1, wherein a ratio of PEDOT:PSS to WPU is 4.5:1-6:1.

3. The conductive polymer composite of claim 1, further comprising dimethyl sulfoxide (DMSO), and a mass fraction of DMSO is 2 wt. %-6 wt. %.

4. The conductive polymer composite of claim 1, wherein a ratio of poly(3,4-ethylenedioxythiophene) to polystyrene sulfonate is 0.05-1.00.

5. A strain gauge, comprising:
a substrate having a surface; and
a strain sensitive layer, wherein the strain sensitive layer is connected to the surface of the substrate;
wherein the strain sensitive layer is made of the conductive polymer composite of claim 1, and the strain sensitive layer has at least four separations arranged in a staggered way and forms bow-like structures, which makes the strain sensitive layer deform more in a first direction than a second direction perpendicular to the first direction.

6. The strain gauge of claim 5, wherein the substrate is made of an elastomer.

7. The strain gauge of claim 5, wherein an area of the at least four separations is A_{H}, an area of the strain sensitive layer is A_{S}, and the following condition is satisfied:
0.2≤A_{H}/A_{S}≤0.8.

8. The strain gauge of claim 5, wherein the strain sensitive layer generates a first strain in the first direction and a second strain in the second direction, and a difference between the first strain and the second strain increases as a number of the at least four separations increases.

9. The strain gauge of claim 5, further comprising an adhesion layer disposed between the substrate and the strain sensitive layer, wherein the adhesion layer is made of WPU.

10. The strain gauge of claim 5, wherein a strain measurement of the strain gauge is up to 400% strain.

11. The strain gauge of claim 5, wherein the strain gauge performs a strain measurement and a torque measurement.

12. A biomedical device, comprising:
the strain gauge of claim 5;
wherein the biomedical device is a smart bandage or an ECG pad.

13. An electronic device, comprising:
the strain gauge of claim 5;
wherein the electronic device is a humidity sensor, a touch sensor, a touch screen or a shear sensor.

14. A method of manufacturing a strain gauge, comprising:
providing a substrate, wherein the substrate has a surface;
performing an etching step to etch a pattern on the surface of the substrate;
performing a coating step to coat the conductive polymer composite of claim 1 onto the surface, which is etched, of the substrate, so as to form a strain sensitive layer on the substrate, and the strain gauge is obtained;
wherein the strain sensitive layer has at least four separations arranged in a staggered way and forms bow-like structures, which makes the strain sensitive layer deform more in a first direction than a second direction perpendicular to the first direction.

15. The method of manufacturing the strain gauge of claim 14, wherein the substrate is made of an elastomer.

16. The method of manufacturing the strain gauge of claim 14, wherein in the etching step, the substrate is etched by CO₂ laser.

17. The method of manufacturing the strain gauge of claim 14, wherein in the coating step, the conductive polymer composite is coated onto the surface by an inkjet printing method, a spreading method or a soaking method.

18. The method of manufacturing the strain gauge of claim 14, wherein the strain sensitive layer generates a first strain in the first direction and a second strain in the second direction, and a difference between the first strain and the second strain increases as a number of the at least four separations increases.

19. The method of manufacturing the strain gauge of claim 14, wherein before the coating step, the method of manufacturing the strain gauge further comprises:
performing an adhesion layer coating step to coat an adhesion layer onto the surface, wherein the adhesion layer is made of WPU.

20. The method of manufacturing the strain gauge of claim 14, wherein after the coating step, the method of manufacturing the strain gauge further comprises:
performing a wiring creating step by applying a magnetic connector or a silver epoxy onto the strain sensitive layer, so as to form a wiring connection to the strain sensitive layer.
